(19) United States
(12) Patent Application Publication
(10) Pub. No.: US 2009/0269834 A1
(43) Pub. Date: Oct. 29, 2009

(54) MICRO REFINERY SYSTEM FOR ETHANOL PRODUCTION

(76) Inventor: Thomas J. Quinn, Los Gatos, CA (US)

Correspondence Address: DERGOSITS & NOAH LLP, Suite 1450, Four Embarcadero Center, San Francisco, CA, 94111, US

(21) Appl. No.: 12/110,158

(22) Filed: Apr. 25, 2008

(51) Int. Cl. C12M 1/36

(52) U.S. Cl. 435/286.2; 435/286.1; 435/286.5

(57) ABSTRACT

The micro refinery first detects the weight of the sugar added to the fermentation tank and then calculates the water needed for fermentation. The feed stock is then inserted into the fermentation tank and the system adds the corresponding volume of water. The control system monitors the weight of the batch and maintains the temperature within a fermentation temperature range so the batch is converted into ethanol. In another mode of operation, discarded alcoholic beverages can be placed in the fermentation tank and processed by the micro refinery to extract the ethanol. The fermented liquid is heated and the ethanol vapors travel through a distillation tube to a membrane separation unit that separates water from the ethanol. The distillation tube has an alignment system that orients the distillation tube vertically automatically. The ethanol from the membrane separation unit is then stored in a storage container prior to use in a vehicle. The micro refinery can blend the ethanol with gasoline to produce any desire ratio of fuel.

### FIELD OF INVENTION

The present invention relates generally to ethanol refinery system used to convert starch or sugar or discarded alcohol into ethanol.

### BACKGROUND OF THE INVENTION

Ethanol fermentation is the biological process by which sugars are converted into ethanol which can be used as fuel for internal combustion engines. Starch or sugar-based feedstocks can be used to produce ethanol or ethyl alcohol. Large fermenters are used to convert sugar and yeast into ethanol. After fermentation, the ethanol is separated from the other fluids in a distillation process. The anhydrous ethanol can be blended with gasoline and then shipped to gasoline terminals or retailers.

A problem with large industrial ethanol fermentation facilities is that the production requires large scale machinery that produces large batches of ethanol. The ethanol must then be blended with additives, transferred to delivery trucks and delivered to gas stations. What is needed is a more convenient system for producing ethanol in smaller batches using machinery that can be easily set up and operated by consumers to fuel their internal combustion vehicles.

### SUMMARY OF THE INVENTION

The present invention is a micro refinery apparatus for producing ethanol from both sugar and recycled alcoholic beverages. The micro refinery includes: a user interface having a graphical display, a processing unit, a fermentation tank, a load cell weight detection system, a temperature control system and a mixing agitator for the fermentation tank, a distillation system, a membrane separation system, a storage tank and a blending and pumping system that can all be mounted within a protective housing. The individual systems are coupled to the processing unit which controls the operations of the systems and provides information and instructions to the user through the graphical display. The controller receives instructions from the user through an input device such as a keypad or other input devices. In an embodiment the controller can be coupled to a computer network which allows a networked computer to monitor the processing of the materials.

The fermentation tank can be made of a rigid plastic or metal or made of a flexible material such as an inflatable structure having a special wall construction and material that are compatible with the fuel. Because the system is intended for consumer use, the micro refinery system can be manufactured in one location and shipped to the end users throughout the world. In order to minimize the size of the system for transportation, the fermentation tank can be compressed for shipping. When the system is installed at the user's site the fermentation tank can be expanded and the system can be coupled to a power supply, a water supply and a fluid drain. Because the system includes a fuel pump, it should be installed at a location easily accessible to vehicles. Set up of the micro refinery requires placement on a level surface and connecting it to a source of water, power and waste water disposal. The machine operates automatically managed by the user through an LCD interface.

When the system is installed at a user's location and the user turns the micro refinery on, the system may go through a start up process to check each system for proper operation. Once the system is ready to begin processing, sugar is first inserted into the fermentation tank. A measuring system utilizes the load cell to detect the quantity of feedstock placed in the fermentation tank based upon the change in weight. A feedstock is then inserted which includes an inexpensive form of yeast and yeast nutrients, into the fermentation tank which is a sealed unit that includes an agitator, temperature control mechanisms. A proper volume of water is automatically mixed with the feedstock so that the fermentation process can begin.

A control system manages the pumps, agitator, valves, sensors and thermoelectric coolers that automatically maintain the proper fermentation environment. A temperature transducer is coupled to the fermentation tank and detects the batch temperature. The temperature control mechanism keeps the batch within a specified temperature range such as 60 to 90 degrees Fahrenheit or a narrower temperature range if necessary. If the batch temperature is below the specified range, the system controller instructs the thermoelectric cooler to heat the batch. Conversely, if the batch is too hot, the system controller instructs the thermoelectric cooler to cool the batch. The heating and cooling can be performed is different ways. In an embodiment, the thermoelectric cooler is coupled to the fermentation tank and the heating or cooling is applied to the batch within the fermentation tank. Alternatively, if heating or cooling is required, the batch is pumped through a thermoelectric coolers radiator which immediately alters the temperature of the fluids passing through. By keeping the temperature within the required temperature range, the fermentation process will take place.

The yeast consumes sugar and converts it into ethanol, carbon dioxide gas and heat. The carbon dioxide is vented from the fermentation tank and the weight of the batch is decreased. The system can detect the weight of the ingredients by monitoring the output signals from the load cells. By detecting the change in weight of the materials over time, the system can identify the status of the fermentation process. For example, the system can predict the weight of a completely fermented batch based upon the weight and type of sugar initially placed in the fermentation tank. When the detected weight of the batch matches or is lower than the predicted fermented batch weight, the system can indicate that fermentation is complete. Alternatively, the system can detect the rate of weight change. Initially, the batch will loose weight quickly because there is a large quantity of sugar available. The rate at which the batch loses weight decreases as the fermentation process progresses. When the fermentation process is complete, the rate of weight loss may stop or be very low. The system can detect the rate of weight change and predict that the fermentation process is complete when the rate change falls below a predetermined set point.

After fermentation, the fermentation tank includes ethanol and water and other residual liquids. The inventive system uses a distillation system to separate the ethanol from the water and other liquids. Over a period of several days, the fermented liquids are slowly pumped from the fermentation tank through a heater which vaporizes the liquids. The ethanol and water vapors are directed to the bottom of a distillation column. Because the boiling points of these materials are different, the ethanol vapor will tend to rise to the top of the distillation tube while most of the water vapor condenses on the tube wall and does not exit the distillation tube. In an embodiment the distillation column is made of Teflon. Ethanol vapors can travel faster upward through the Teflon column wall because Teflon will not alter the temperature inside the column wall as vapors rise through the heating column process.

Because the system can be mounted in any location, the system includes a vertical alignment system for the distillation tube. In an embodiment, a gimbaled mechanism is coupled to the upper half of the distillation tube. Because most of the weight is below the gimbaled mechanism, the distillation tube will automatically tend to rotate into vertical alignment. In an embodiment, the system may have a locking mechanism that only allow free rotation during an alignment process. After the alignment is performed, the system may lock the distillation tube in place to prevent rotational movement during the distillation process. In other embodiments, the system may accelerometers that detect the direction of the gravity and motor actuators that may utilize fine thread screw drives to rotate the distillation tube into vertical alignment. In yet another embodiment, ethanol quality from the distillation tube may be sampled at different distillation tube alignment angles and the distillation tube may be positioned at the angle that produces the best separation of water and ethanol resulting in a higher purity of ethanol. The separation efficiency can be sampled at the outlet of the distillation tube or at the outlet of the separation membrane.

Vapors exiting the distillation tube are passed directly to a separation membrane that separates the ethanol from the other fluids. The system does not require refluxing or recycling of content. The membrane can be damaged by the thermal shock if the membrane is at a low ambient temperature and then exposed to hot vapors too quickly. In order to prevent damage to the membrane from the hot vapors, the system may include a pre-heating mechanism that detects the membrane temperature and slowly heats the membrane before it is exposed to the hot vapors from the distillation tube. The membrane has small holes that allow the smaller water molecules to pass but not the larger ethanol molecules that stream through the exit port. A number of heat exchangers and thermal electric coolers convert the ethanol and water vapors back into liquids and make this process energy efficient. The water is recycled and the fuel grade ethanol is available for use.

In an embodiment, the micro refinery stores the ethanol as well as regular gasoline. The user interface may have controls that allow the user to control the blending ratio of the ethanol to gasoline. The outlets of the pumps coupled to the gasoline and ethanol tanks are coupled and the flow rates of the pumps are set to produce the desired mixture of ethanol and gasoline. When fuel is needed, the blended fuel is dispensed just like at a gas station through a hose and nozzle.

### BRIEF DESCRIPTION OF THE DRAWINGS

FIG. 1 is a side view of an embodiment of the micro refinery system;

FIG. 2 illustrates a thermoelectric mechanism;

FIG. 3 is a graph showing the change of the batch weight over time;

FIG. 4 illustrates a gimbaled mechanism used for vertical alignment of the distillation tube;

FIG. 5 illustrates an embodiment of a vertical alignment system and gravity detection system used for vertical alignment of the distillation tube;

FIG. 6 illustrates an embodiment of a locking mechanism used to prevent rotation of the distillation tube;

FIG. 7 illustrates an embodiment of a locking mechanism used to prevent rotation of the distillation tube;

FIG. 8 illustrates a cross section of a porous membrane used to separate water and ethanol;

FIG. 9 illustrates the system controller and the connections to the sensors and control mechanisms of the micro refinery system; and

FIG. 10 is a side view of a collapsible embodiment of the micro refinery system.

### DETAILED DESCRIPTION

The present invention is for a micro ethanol refinery system that functions as a sugar fermentation tank to produce ethanol. The micro ethanol refinery system processes discarded sugar and starch matter as well as alcohol in the tank that feeds a distillation system for conversion into ethanol.

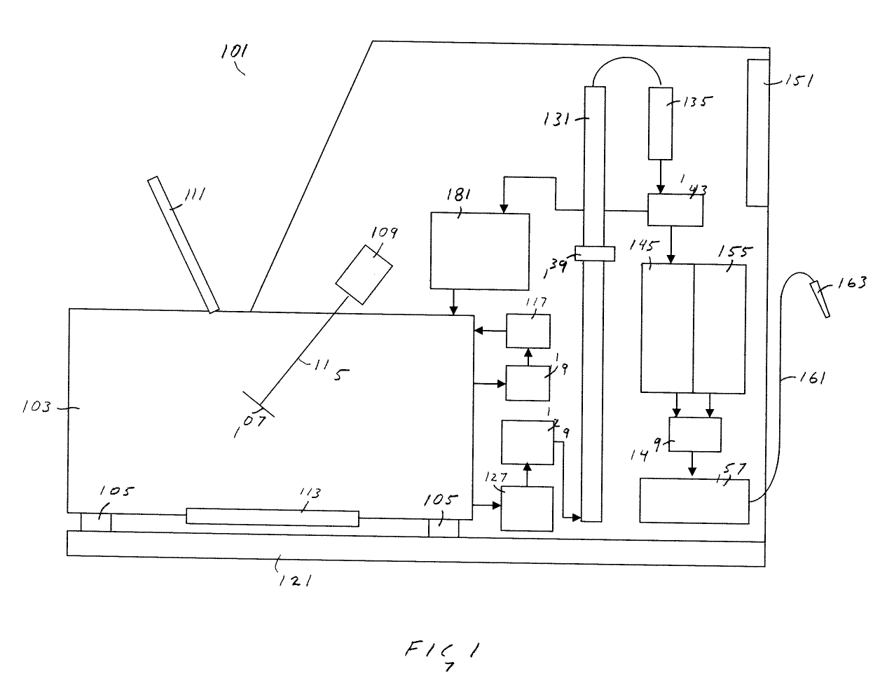

With reference to FIG. 1, a diagram of the micro ethanol refinery system 101 and system components is illustrated. In an embodiment, the system 101 includes a frame 121 that is supported by a plurality of locking casters which allow the system to be rolled to the desired location and locked in place by locking the rotation of the casters. The frame 121 may be adjustable so that the casters can be retracted and the frame can rest on legs when moved to the desired location. The castors or legs may also be adjustable in length so that each can be adjusted to equally support the frame and the system can be adapted for uneven surfaces. In other embodiments, the frame 121 can be placed directly on the ground or supported by pilings, foundations or any other support structures.

The components of the expanded micro refinery system 101 will be described with reference to FIG. 1. In an embodiment the fermentation tank 103 rests on one or more load cells 105 that detect the downward force and produce corresponding electrical output signals. The load cells 105 are coupled to a system controller 151 that monitors the weight of the tank 103 and all contents within the tank 103 throughout the ethanol conversion process. The load cell 105 output signals are proportional to the detected weight. In an embodiment, the system controller 151 can go through a calibration process which detects the weight of the empty tank 103 and stores the empty tank weight as an offset value. The offset value can then be subtracted from any detected weight so that the system controller 151 can detect the weight and quantity of materials that are inserted into the tank 103. The fermentation tank 103 calibration process may be repeated each time a batch of materials is processed.

The system controller 151 may provide a display and/or audio instructions which may indicate the sequence of materials and quantities to be inserted based upon the estimated quantity of ethanol to be produced. For example in an embodiment, a user may input the quantity of ethanol desired. The system then calculates the expected quantities of materials required to produce the desired quantity of ethanol and instructs the user to insert specific quantities of sugar and feedstock. To start the fermentation process, the lid 111 is opened and a specific ratio of sugar and feed stock are inserted into the tank 103.

In an embodiment, the sugar is the first material added to the fermentation tank 103. The weight of the sugar is detected by the system controller 151 and the corresponding volume of water is determined. After the sugar has been added, the system controller 151 can instruct the user to insert the feedstock. The system controller 151 can detect the weight of feedstock and provide instructions and information regarding the quantity of feed stock to add to the fermentation tank. The system controller 151 can detect the weight of the materials being inserted and may provide instructions to the user such as: add more, slow the rate of insertion in preparation to stop and stop. The system controller 151 may have a visual display that indicates the volume of materials added to the tank so the user knows when to stop adding materials to produce the desired volume of ethanol. The system controller 151 may also provide feedback if errors are made. For example, if the system controller 151 detects that too much sugar was added, the system may compensate for this error by increasing the feedstock needed in the fermentation tank 103 for the extra sugar.

In another embodiment, the sugar and feedstock are stored in containers that are coupled to the fermentation tank 103 and the control system 151 can control the flow of materials into the fermentation tank 103 so that the insertion of the sugar and feedstock is automated. When the proper volume and ratio of feedstock and sugar have been inserted into the fermentation tank 103, the lid 111 is closed. The lid 111 may have a locking mechanism to prevent the addition of any other materials to the tank 103 until after processing is completed.

As discussed, the system controller 151 detects the quantity of sugar in the fermentation tank 103 and calculates the corresponding volume of water for the fermentation process. The system can automatically add the volume of water required for fermentation processing to the tank 103. The proper volume of water can be detected based upon a metered flow of water from a water storage tank 181. Alternatively, the system controller 151 can detect the weight of the water and calculate the volume of water added based upon the known volumetric weight. The system controller 151 is coupled to a valve between the water tank 181 and the fermentation tank 103. The system controller 151 can open the valve to cause water to flow into the tank 103 and when the proper volumetric weight change is detected, the system controller 151 can shut the valve. In other embodiments, the water can be added to the fermentation tank 103 manually.

While the detection of the materials placed in the tank 103 has been described with the use of force transducers 105 and a calibration process, in some cases additional correction factors may be required. For example, ideally the sugar and feedstock are dry, however humidity and moisture can cause the sugar and feedstock to absorb some water which will cause inaccurate detection of the volumetric weights. In order to compensate for the absorbed moisture the system controller 151 may apply a correction factor that determines the weight of the absorbed water. The system may detect the humidity and temperature in the sugar and feedstock storage containers. Based upon this information, the volumetric weight of the sugar and feedstock materials can be accurately calculated. In an embodiment, the system may weigh a specific volume of the materials just prior to inserting the materials into the tank. The measured volumetric weights are then stored in memory and used to calculate the volume of materials based upon weight signals from the transducers 105.

With the proper mixture of water, feedstock and sugar in the fermentation tank 103 the system can mix the ingredients by rotating the agitator 107 to mix the materials. In an embodiment, a motor 109 is used to rotate shaft 115 coupled to an agitating element 107. The agitating element 107 can be an elongated angled mixing blade that circulates liquids in the tank when rotated. The rotation of the agitating element 107 causes the water, feedstock and sugar to be mixed. The mixing may be required to cause the yeast in the feedstock to come in contact with the sugar and nutrients required for fermentation. While a single agitator 107 is illustrated, in other embodiments multiple agitators can be used to mix the materials and prevent clumping of the sugar and feedstock in the corners of the tank 103.

In an embodiment, the control system 151 may detect the proper mixture of the materials by the rotational resistance of the agitator 107. A low resistance indicates that the agitator 107 is only in contact with water while a higher resistance may indicate that the agitator 107 has contacted a clump of sugar or feedstock. Thus, during the mixing process, the rotational resistance is an indication of the status of the mixing. The materials may be properly mixed when the rotational resistance is steady and corresponds to a proper resistance range for the mixture. In an embodiment, the control system 151 measures the rotational resistance of the agitator 107 by monitoring a torque transducer coupled to the shaft 115 between the motor 109 and agitator 107. Alternatively, the control system 151 can measure the rotational velocity of the motor 109 for a given applied power. A higher rotational velocity indicates a low viscosity and a lower rotational velocity indicates a higher viscosity. Once the proper mixed viscosity is detected, the materials are properly mixed and the rotation of the agitator 107 can be stopped or run periodically during the fermentation process.

During the fermentation process, the yeast absorbs the sugar when diluted in water. This reaction produces 50% ethanol and 50% CO₂ by the end of the fermentation process. The chemical equation below summarizes the conversion:

C₆H₁₂O₆ (Glucose)→2CH₃CH₂OH (Ethanol)+2CO₂+heat

A requirement of fermentation is proper temperature control to keep the ingredients within a proper fermentation temperature range. If the yeast temperature is too cold the yeast can become dormant and fermentation is slowed and if the temperature is too high the yeast can be killed. There are various types of yeast, some of which have a high temperature tolerance. The internal temperature of the fermentation tank 103 should be between about 60 and 90 degrees Fahrenheit to preserve yeast culture life. In order to increase the speed of fermentation, the temperature may be at the higher end of the yeast tolerance temperature range.

In an embodiment, the system 101 also includes a thermoelectric mechanism 113 that can be coupled to the fermentation tank 103. The thermoelectric mechanism 113 is powered by a DC electrical power supply and maintains the optimum processing temperature within the tank 103. In order to provide uniform temperature control, a plurality of thermoelectric mechanisms 113 can be attached to various sections of the tank 103. In an embodiment, the system controller 151 is coupled to the thermoelectric mechanism 113 and a temperature transducer mounted within the fermentation tank 103. The system controller 151 receives a signal corresponding to the internal tank temperature from the temperature transducer and determines if the fermentation tank 103 is within the proper temperature range if the batch needs to be heated or cooled. As discussed above, the fermentation process produces heat, so in some cases heating of the tank 103 may not be required. If the system detects that the fermentation tank 103 is too cold, the system controller 151 applies direct current electrical power to the thermoelectric mechanism 113 in the heating mode of operation or reverses the polarity of the electrical power to the thermoelectric mechanism 113 in the cooling mode. The system controller 151 can also turn the power to the thermoelectric mechanism 113 off when the fermentation tank 103 temperature is within the proper temperature range for fermentation.

In another embodiment, the system may utilize a pump 119 that pumps the batch through a thermoelectric radiator 117 that is separate from the fermentation tank and then returns the batch to the fermentation tank. If the system controller 151 detects that the batch is too cold, the pump 119 is actuated to pump the batch through the thermoelectric radiator 117 which is controlled by the controller 151 to heat the batch. Alternatively, if the system controller 151 detects that the batch is too hot, the pump 119 is actuated to pump the batch through the thermoelectric radiator 117 which is controlled by the controller 151 to cool the batch. The outlet of the thermoelectric radiator 117 can be coupled to the fermentation tank 103 so that all thermally processed batch materials are returned to the fermentation tank 103.

In an embodiment, the system can be used in a wide variety of environments and has the ability to produce ethanol in a wide range of ambient conditions. This requires the cooling of the fermentation tank in hot regions and seasons and heating of the fermentation tank in cold areas and seasons. A larger number of thermoelectric mechanisms 113 can be used in the system based upon more extreme expected ambient temperatures. In an embodiment, the user can simply purchase and install additional thermoelectric mechanisms 113 to compensate for the more extreme ambient conditions. It is also possible to reduce the effects of extreme ambient temperatures by placing the micro refinery system within a protective enclosure.

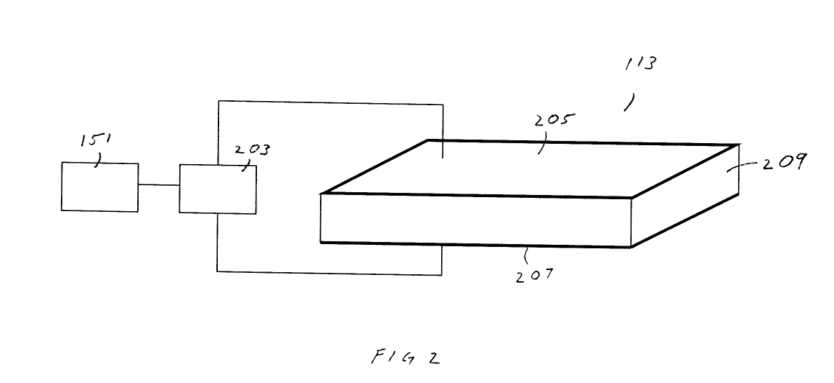

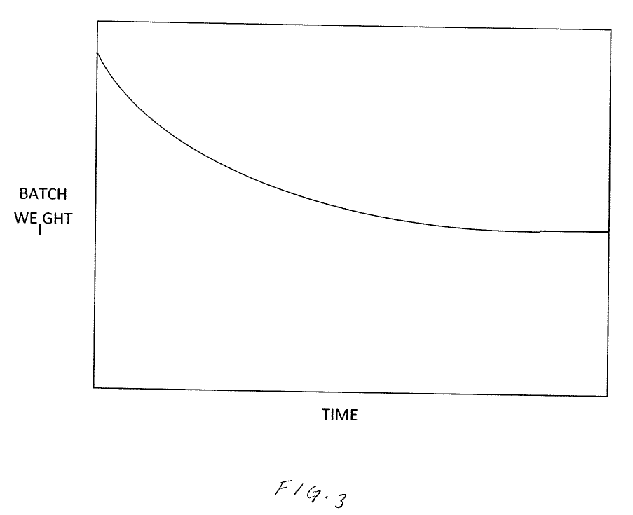

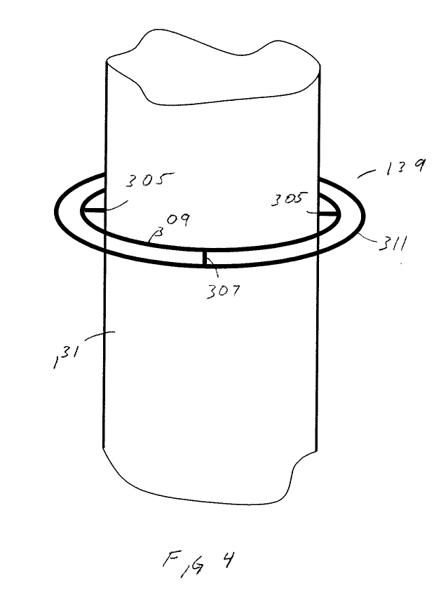

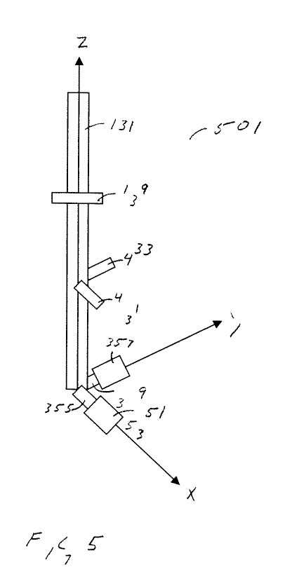

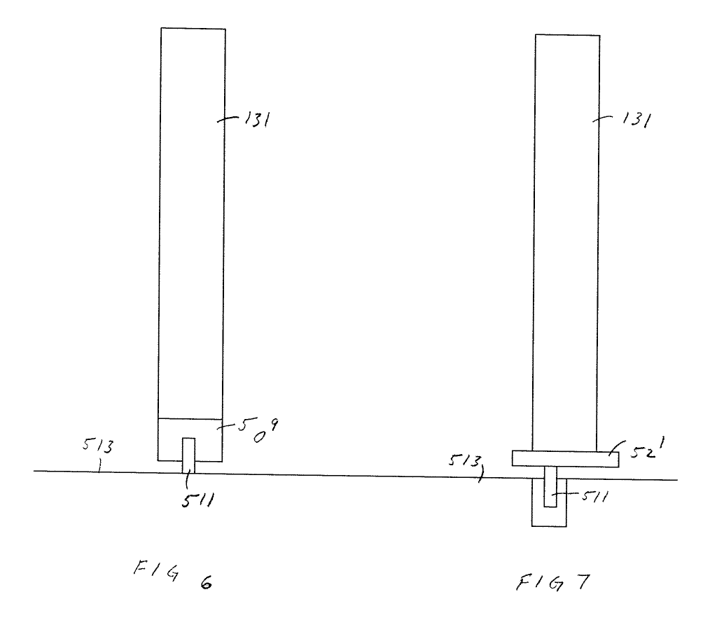

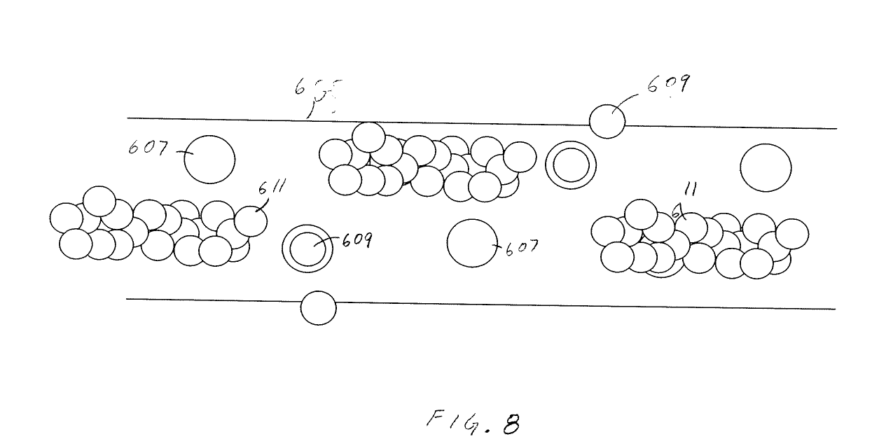

With reference to FIG. 2, in an embodiment the thermoelectric heating and cooling mechanism 113 can have two metal plates 205 and 207 that surround a ceramic core 209. When electrically charged, the plates 205 and 207 oscillate creating a cool side 205 and hot side 207. In an embodiment, the two plates 205 and 207 always maintain a 69 degree temperature difference between the two sides of 205 and 207. The voltage is applied to the plates 205 and 207 by DC power source 203 which is controlled by the system controller 151. The heating or cooling output of the thermoelectric mechanism 113 can be controlled by reversing the polarity of the applied electrical power from the power source 203. Since thermoelectric mechanisms 113 are small in size, about 2 to 4 inches in diameter, the thermoelectric mechanisms 113 are ideal for small cooling and heating applications such as the batch materials in the fermentation tank.

The thermoelectric mechanisms 113 can be mounted on the fermentation tank 103 walls or, as discussed above with reference to FIG. 1, the thermoelectric mechanisms can be configured as a thermoelectric radiator 117. The fermentation liquid can be pumped through a thermoelectric radiator 117 to provide heating and cooling. Thus, the thermoelectric heating and cooling mechanism 113 and thermoelectric radiator 117 can cool the batch fermentation tank or heat the batch through the system controller 151 by reversing the DC polarity applied to the thermoelectric mechanisms 113 and thermoelectric radiator 117.

In a preferred embodiment, the fermentation tank 103 holds about 200 gallons of liquid. The thermoelectric mechanisms 113 are practical for small fermentation batches in this liquid volume range, but lack enough thermal energy to perform thermal control of larger commercial fermentation processing. For these reasons, the thermoelectric mechanisms can be used with the inventive system to the control the temperature of about 200 gallons of liquid but are not suitable for temperature control of a larger 1,000+ gallon commercial fermentation processing tank.

A problem with the fermentation process is that it is not a predictable process. The time required to complete the fermentation process will vary depending upon the purity of the sugar and yeast, as well as the batch temperature. One way to monitor the fermentation progress is by monitoring the change in weight of the fermenting liquid. During fermentation, the sugar is converted into ethanol and CO₂ which is vented out of the fermentation tank 103. Thus, the venting of the CO₂ results in a weight reduction of the batch. In an embodiment, the force sensors 105 are used to periodically or continuously check the weight of the batch during the fermentation process. As CO₂ is vented from the fermentation tank 103, the batch gets lighter. An initial weight of the batch can be determined and stored in memory. Any change in the batch weight will change depending upon the rate of CO₂ venting from the fermentation tank 103. The system controller 151 can determine that the fermentation process is complete when the weight of the batch is reduced by a known percentage or to a predetermined weight based upon the original quantity of sugar in the batch. Alternatively, the system controller 151 can determine that the fermentation process is complete when the rate of weight reduction slows or stops indicating that less CO₂ is being vented. When the weight reduction stops after all the sugar has been processed or if CO₂ is no longer being emitted from the batch, the fermentation of the batch is complete.

As discussed above, the force sensors 105 can be used for detecting an initial start weight of the sugar loaded into the tank 103 at the beginning of the fermentation process. The weight can then be detected periodically by sampling the force sensors 105 at timed intervals. By monitoring the weight of the batch over time, the rate of weight change can be determined by the system. The processor can use the information to determine the stage of the batch in the fermentation process. For example with reference to FIG. 3, a graphical representation of the weight of the batch over time is illustrated. At the beginning of the process, the weight of the batch drops fairly quickly. As the conversion of the sugar to ethanol progresses, the rate at which the weight decreases slows. Eventually, the weight change becomes very low indicating that the fermentation process is complete. In an embodiment, the system controller can determine that one or more of the fermentation complete indicators has been satisfied and cause the fermented batch to be distilled.

Although the fermentation tank 103 has been described above for fermenting sugar and feedstock, the inventive system also has the ability to process different materials and can extract ethanol from recycled alcoholic beverages such as beer, wine and other alcohol products. The user can select the function of the micro refinery system as either a sugar fermentation tank or a processor of discarded alcohol. In the sugar fermentation mode, the micro refinery system ferments the sugar to create alcohol as described above. In the alcohol recycling mode, the alcoholic products also go into the fermentation tank prior to being processed by a distillation system for conversion into ethanol. The multi-function design provides a market advantage for recycling either sugar or discarded alcohol commonly found at bar restaurants or wineries.

After the fermentation of the sugar is completed, it is possible to add the alcoholic liquids to the fermentation tank. When the fermentation process is complete, the processor can indicate that alcoholic beverages can be added and the lid can be unlocked. Because the reaction of the yeast has converted much of the liquid into carbon dioxide, the volume of liquids in the fermentation tank will decrease after fermentation is complete which allows room for to recycle the alcoholic beverages. The micro refinery will then separate the ethanol from the batch as well as the alcohol from the discarded beverages from the other liquid components.

In an alternative mode of operation, the micro refinery can be used to convert alcoholic beverages into ethanol. In many wineries, bars and restaurants, alcoholic beverages are thrown away or poured down the drain. Rather than disposing of these liquids, they can be processed by the micro refinery by simply poured them into the fermentation tank 103. In this mode of operation, the fermentation tank can be empty and since the alcoholic beverages contain ethanol the fermentation processes described above are not necessary. The micro refinery only performs the task of separating the ethanol from the other liquids mixed with the beverages as described below.

When the fermentation process is completed or alternatively after alcoholic beverages are inserted into the fermentation tank 103, the system controller can begin the distillation processing. A distillation system is used to separate the ethanol from water and other liquids. Distillation is a method of separating chemical substances based on the differences in their boiling points in a liquid mixture. Since alcohol boils at a lower temperature than water and other liquids, the ethanol will vaporize first and will tend to remain in vapor unit until it exits the top of the distillation tube. In contrast, other liquid components including water and other impurities have a higher boiling point and will tend to remain in liquid form or condense at a lower temperature than the ethanol vapor.

In an embodiment, the distillation system of the present invention includes a pump 127, a heater 128, a distillation tube 131 and a gimbaled mechanism 139 that is used to position the distillation tube 131 in a vertical orientation. When the fermentation is complete, the control system 151 controls the pump 127 to pump the liquids in the fermentation tank 103 through the heater 129 to cause the liquid to boil. The vaporized liquid is directed to the bottom of the distillation tube 131. As the vapors travel higher through the distillation tube 131, the alcohol molecules separate from water molecules and eventually exit the upper part of the column. If water and other non-ethanol liquids vaporize, these vapors will tend to be condensed on the sides of the distillation tube as they cool in the distillation tube 131. The condensed liquids may then adhere or drip down the inner walls of the distillation tube 131 rather than exiting the top of the tube 131. The distillation system may also include one or more temperature sensors which monitor the vapor temperature and control the heater 128 to produce vapor at an optimum separation temperature. Excessive heat will cause a faster vapor velocity resulting in more water exiting the distillation tube 131, while a low temperature vapor temperature will result in a low flow of ethanol from the distillation tube 131.

In order to enhance the separation of ethanol and water, the distillation tube 131 may include additional components that increase the vapor and liquid contact which improve the separate performance. Within the distillation tube 131, there are a number of separation stages where the liquid and vapor phases of the liquid are in equilibrium. Each component of the liquid mixture will have a different separation stage. As the vapor travels upwards in the distillation tube 131, the pressure and temperature decreases at each succeeding stage. In an embodiment, the distillation tube 131 includes a series of plates which are horizontal perforated structures that are placed at the separation stages within the distillation tube 131 to improve the separation of the corresponding liquid component. The plates used in the present invention may be custom designed for the inventive distillation tube 131 and may be unique in design due to the small size of the distillation tube 131.

Another means for enhancing separation is the use of a packing material instead of plates within the distillation tube 131. The packing material can be small random objects or structured packing objects that are placed in the distillation tube 131 at the separation stages along the length of the distillation tube 131. The packing material functions like the plates described above and increase the vapor component separation. The packing results in less of a pressure drop than plates as the vapors travel through the distillation tube 131 and therefore packing is more efficient than plates. However, the packing also more susceptible to plugging from contaminants and more difficult to clean than plates. In various embodiment of the present invention, the distillation tube 131 may include plates or packing.

The distillation tube 131 can be made of various materials including glass, ceramics, metals, plastics and other high temperature resistant composite materials that can withstand a working temperature of more than 200 degrees Fahrenheit. Because the thermal properties of the distillation tube will influence the efficiency of the ethanol separation, some distillation tube materials will perform better than others. In an embodiment, the distillation tube 131 can be made from Teflon which provides additional safety and enhances ethanol purity performance. Teflon can provide a natural heat shield by acting as a thermal insulator that may prevent the transmission of high heat generated inside the column to the outer surfaces of the distillation tube 131 that may con into contact with the system operators. Teflon also increases the distillation power efficiencies because it does not act as a thermal conductor to transfer, lose or dissipate heat to the outside. The ethanol vapors can travel faster upward through the column wall undisturbed or unrestricted by the column walls because Teflon will not alter the temperature inside the column wall as vapors rise through the heating column process. Thus, there is a very low thermal variation through the cross section of the distillation tube 131 and the ethanol vapor distillation processing is also more efficient.

The distillation process requires that the distillation tube 131 be in a perfect vertical alignment. The vapors slowly rise vertically straight up and the flow path is preferably undisturbed by sidewalls as the vapors travel up through the center of the distillation tube 131 and out from the top. If the distillation tube 131 is out of alignment, the rising vapors will run into the side of the tube 131 resulting in condensation of ethanol vapors and reducing the efficiency of the distillation system. Similarly, water vapor rising on the side wall tilted away from vertical may not condense on the sidewalls resulting in a reduction in the separation of the water and ethanol. Thus, perfect vertical alignment is necessary for the high efficiency distillation.

Perfect vertical alignment can be difficult because the inventive micro refinery system is intended to be used at homes and small businesses where a perfectly level surface to place the micro refinery may not be available. To compensate for uneven surfaces, the inventive system may have an alignment system which vertically aligns the distillation tube mechanisms within the system. The legs of the frame may be adjustable in height with adjustable screws so that the weight of the system is evenly distributed and the frame is very stable. With reference to FIG. 4, a gimbaled mechanism 139 is shown which supports the distillation tube 131 and allows the tube 131 to naturally rotate into vertical alignment. The gimbaled mechanism 139 includes an inner pivot 305 that couples the distillation tube 131 to a ring 309 that surrounds the tube 309 and an outer pivot 307 that couples the ring 309 to fixed support 311 that is coupled to the frame. The pivots 307 and 309 may include low friction bearings or bushings which allow the pivots 307 and 309 to rotate with very low friction. The gimbaled mechanism 139 is mounted above the center of gravity of the distillation tube 131 so that the weight of the distillation tube 131 below the gimbaled mechanism 139 will cause the tube 131 to automatically self align in a vertical orientation. If the fixed support 311 is rigidly mounted to the frame, the distillation tube 131 automatically rotate to vertical alignment.

In other embodiment with reference to FIG. 5, the distillation tube 131 may have a closed loop alignment and an electronic vertical detection system 501 that are coupled to the system controller 151 and used to detect a vertical direction and align the distillation tube 131 vertically. The distillation tube 131 may be arranged in an X, Y, Z coordinate system with the tube 131 extending in the direction of the Z axis. The closed loop alignment system may include a first accelerometer 431 aligned with the X axis and a second accelerometer 433 aligned with the Y axis that are coupled to the distillation tube 131. When the first and second accelerometers 341 and 433 are horizontally oriented and the distillation tube 131 is in a vertical position, the gravitational acceleration is not detected because the force is perpendicular to the acceleration detection. However, the first or second accelerometers 431 and 433 will detect some gravitational force if they are not perfectly horizontal. Thus, if the controller detects an acceleration signal from the first and/or second accelerometers 431 and 443, an adjustment to the alignment is necessary.

The closed loop alignment system can include a first motor driven actuator 351 coupled to a fine thread screw drive 355 that moves the distillation tube 131 in rotation about the Y axis of rotation. Movement of the tube 131 about the Y axis is only detected by the first accelerometer 341. The closed loop alignment system can also include a second motor driven actuator 357 coupled to a second fine thread screw drive 359 that moves the distillation tube 131 in rotation about the X axis of rotation. The movement of the tube 131 about the X axis is only detected by the second accelerometer 341. In this embodiment, the system controller 151 detects the gravitational signals from the accelerometers 341 and 443 and provides the necessary adjustments to the first and second motor driven actuators 351 and 357 to cancel out the misalignment. The closed loop system can detect when the distillation tube 131 is aligned and stop all movement to hold the tube 131 in alignment. The weight of the distillation tube 131 may be supported by the gimbaled mechanism 139.

While vertical alignment can result in the optimum distillation efficiency, it is also possible to determine the optimum distillation tube 131 angle empirically. As discussed above, the alignment of the distillation tube 131 is based upon the assumption that a vertical alignment produces the highest efficiency and best separation of water and ethanol vapors. In an embodiment, the alignment of the distillation tube 131 is based upon the detected efficiency and separation of water and ethanol vapors. The vapors from the outlet of the distillation tube 131 can be sampled and the ratio of ethanol to water can be detected for various distillation tube 131 angles. The sampling can be performed by optical sensors and other fluid flow sensor that can accurately determine alcohol content.

The methodology used to determine the optimum distillation tube angle can be based upon a two dimensional grid with each cell representing different X angles and Y angles combinations. The system can determine the ethanol separation for each grid cell and determine the set of four cells that have the highest ethanol separation values. The system can then subdivide the set of four cells into a narrower range of grid cells and repeat the ethanol separation detection sampling to further narrow the optimum distillation tube 131 angle. This process can be repeated until the optimum distillation tube 131 angle is determined. This optimum angle information can be stored in memory so that if a deviation is detected, the system can restore the distillation tube 131 to the optimum angle.

In some cases it may be desirable to lock the distillation tube 131 in place and prevent rotation. In an embodiment, the vertical alignment system includes a locking mechanism that prevents the distillation tube from rotating. This can be useful to secure the tube 131 after the vertical alignment has been set so that if the apparatus is bumped, the distillation tube 131 will not respond by swinging. The locking mechanism can also be used during transportation of the device to prevent damage to the distillation tube.

Various mechanisms can be used to lock the distillation tube. For example, a movable post may be used to prevent rotation of the distillation tube. With reference to FIG. 6, in an embodiment the post 511 is coupled to the bottom of the distillation tube 131. When the post 511 is extended it contracts a rigid surface 513 which may be attached to the frame. The friction between the end of the post 511 and the rigid surface 513 prevents the distillation tube 131 from rotating. The post 511 may be extended and retracted by a solenoid device 509 which is coupled to the controller. The post 511 may be coupled to a spring so that it is normally extended and the distillation tube 131 is normally locked. When the user wishes to vertically align the distillation tube, the control system performs the alignment by retracting the post 511 which allows the distillation tube 131 to rotate. Once the tube 131 has assumed a vertical position, the post 511 is extended against the rigid surface 513 to lock the distillation tube 131 in position.

In other embodiments, different locking mechanisms can be used to prevent the distillation tube 131 from moving. For example with reference to FIG. 6, a perpendicular base plate 521 can be mounted to the bottom of the distillation tube 131 and extendable post 511 can extend from the fixed surface 513 and contact the base plate 521 to lock the distillation tube 131 in place. When post 511 is retracted, the tube 131 is free to rotate.

Since the tube 131 is easily moved out of alignment, any weight attached to the tube 131 must be symmetrical with the center of gravity of the weight aligned with the vertical axis of the distillation tube 131. As illustrated in FIG. 1, the distillation tube 131 is coupled to other components of the micro refinery system. In an embodiment, the distillation tube 131 is disconnected from the other system components during the alignment process so that they do not create alignment errors. After the distillation tube 131 has been aligned and locked in place, the distillation tube 131 is connected to the other system components. The distillation tube 131 of the inventive micro refinery device is preferably made out of heat and fuel compatible plastic so the weight can be reduced for shipping. The gimbaled distillation tube of the claimed invention is substantially different than the large stainless steel commercial distillation columns that are permanently bolted in place and are not movable.

The hot ethanol vapor from the distillation tube is sent directly to the membrane system which separates water molecules from the ethanol molecules. The vapor does not require refluxing or recycling of content. In large industrial distillation systems, refluxing or recycling can be required such that a portion of the liquid product exiting the top of a distillation column be returned to an upper wall of the distillation column. The returned reflux liquid flows down the sidewalls of the column to provide cooling and condensation of the vapors traveling up the column. Because the inventive system monitors and maintains the vapor heat and the column alignment for optimum water and ethanol separation, refluxing or recycling is not required.

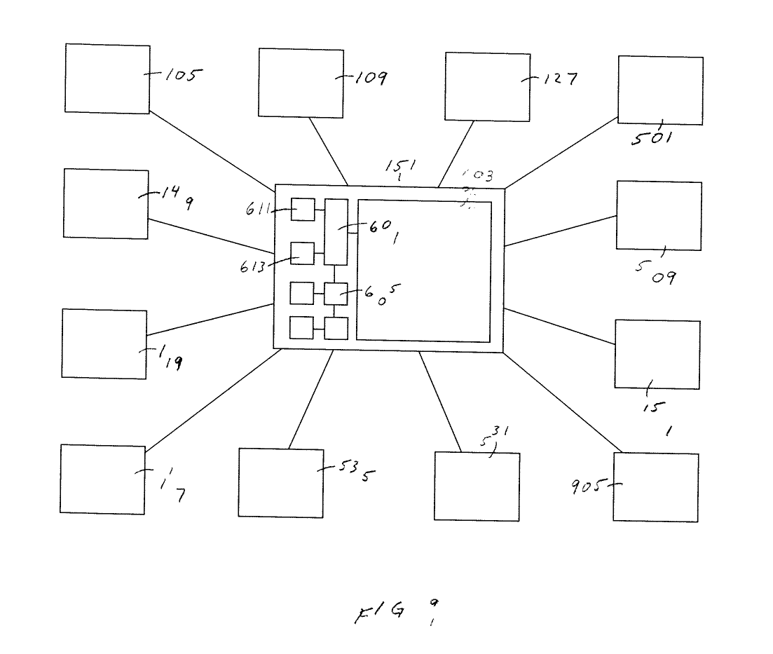

The membrane is made of ceramic, glass or very course materials. With reference to FIG. 8, the membrane 605 has many small holes 607 which allow water molecules 609 to pass through but are too small for the ethanol molecules 611 to pass through. The higher pressure of the vapor causes the smaller water molecules 609 to flow through the holes 607. By passing the vapor through the membrane 605, the water molecules 609 are separated and substantially pure ethanol molecules 609 exits through the membrane system.

A potential problem with the porous membrane is that the membrane materials can be susceptible to this thermal damage. In particular, “thermal damage” of the membrane can occur if the temperature of the ethanol vapor is substantially hotter than the membrane. For example, the membrane may be at ambient temperature and then immediately exposed to hot ethanol vapor resulting in damage. To prevent thermal damage of the membrane a micro controlled warming system is used to pre-heat the membrane to insure the membrane temperature is suitable for processing the hot vapor. In an embodiment, the temperature of the membrane is detected by a thermocouple attached to the membrane system. As the control system directs the flow of fluids out of the fermentation tank through to the heater and distillation tube, it detects the temperature of the membrane before the hot vapors are directed to the distillation tube. If the membrane is cold, the system controller can activate a heating element and monitor the membrane temperature. As the membrane temperature increases, the control system may have a thermostatic setting to prevent over heating of the membrane by the heater. When the membrane temperature is pre-heated to a safe temperature, the control system can allow hot vapors to flow through the distillation tube to the membrane. Once the hot vapors are flowing through the membrane, the membrane will be heated by the vapors and power to the heating element can be removed.

With reference to FIG. 1, after passing through the membrane 135, the separated water can be drawn through a vacuum 143. The water can condense and flow into the water storage tank 181 used to fill the fermentation tank 131. In contrast, the ethanol exits the membrane system 135 and vacuum 143 and then may flow through a thermoelectric cooler which causes the ethanol to condense into a liquid. The liquid ethanol then flows into a storage tank 145 where it is stored before being mixed with gasoline. An ultrasonic sensor coupled to the storage tank 145 can detect the liquid ethanol level within the storage tank 145 and provide this information to the system controller 151. In an embodiment, the system controller 151 can detect when the ethanol storage tank 145 is full and stop the distillation process until there is available space in the storage tank 145.

With ethanol in the storage tank, the user can select the mix ratio of ethanol and gasoline or other fuels. The type of blend ratio can depend upon the type of vehicle being fueled. The use of pure ethanol in internal combustion engines is only possible if the engine is designed or modified for that purpose. However, ethanol can be mixed with gasoline in various ratios for use in unmodified automobile engines. In the United States, normal cars designed to run on gasoline may only be able to use a blended fuel containing up to 15% ethanol. In contrast, U.S. flexible fuel vehicles can use blends that have less than 20% ethanol or up to 85%. The ethanol fuel blend is typically indicated by the letter “E” followed by the percentage of ethanol. For example, typical ethanol fuel names include: E5, E7, E10, E15, E20, E85, E95 and E100, where E5 is 5% ethanol and 95% gasoline, etc.

In an embodiment, the inventive micro refinery can mix the ethanol stored in the ethanol storage tank 145 with gasoline that is stored in a gasoline storage tank 155 in any ratio set by the user through the system controller 151. The control system includes a user interface which allows the user to select the desired fuel blend ratio. The system may include a lock that prevents the fuel mixture setting to exceed the maximum or minimum allowable ethanol percentage for the vehicle. Once the fuel mixture has been selected, the user can use the micro refinery functions like a normal gasoline pump. The user removes the nozzle 163 from a cradle on the micro refinery and places it in the tank filler of the vehicle. A lever coupled to the nozzle 163 is actuated to start the pumps 149 which cause the fuel to flow from the tanks 145 and 155 through the hose reel 157, the hose 161 and nozzle 163 to the tank filler of the vehicle. The system will run the ethanol and gasoline pumps 149 at different flow rates to produce the specified fuel ratio. The nozzle 163 will detect when the vehicle tank is full and automatically stop the flow of fuel through the nozzle 163. When the vehicle tank is full, the user places the nozzle 163 back in the cradle and replaces the cap on the fuel filler to end the filling process. With the ethanol tank 145 at least partially drained, the system can begin to produce more ethanol.

After processing, the system may also need to be cleaned. This can be accomplished by spraying the fermentation tank with pressurized water which will remove particulates from the tanks. The system may remove the waste liquids from the fermentation tank and the distillation system. In an embodiment a valve is opened to allow the waste liquids to drain from the system through a drain hose. Since all of the volatile materials have been removed, the waste materials can be poured down into public drainage systems.

As discussed above with reference to FIG. 9, the system controller 151 is connected to the various sensors, system controls, displays and user interfaces. With reference to FIG. 9, a block diagram of the system controller 151 is illustrated. The controller 151 includes a central processing unit (CPU) 601, a visual display 603 which may also be an input device, user input mechanisms 605 such as buttons, key pads, etc. In order for the CPU 601 to communicate with analog devices such as motors and sensors an analog to digital converters 611 and digital to analog converters 613 are required. The analog to digital converters 611 are used to convert analog data signals into digital signals that can be interpreted by the CPU 601 and the digital to analog converters 613 are used to convert the digital control signals from the CPU 601 to the analog devices.

In an embodiment, the controller 151 is coupled to the load cells 105 to detect the weight of the materials placed in the fermentation tank 103. The load cell 105 outputs can be monitored to detect the volume of materials placed in the tank and detect the progress of the fermentation processing. The controller 151 can also be coupled to the agitator motor 109 and a rotation or torque transducer to detect the mixing status of the batch materials. The controller 151 can also be coupled to the pump 127 to start and control the flow of liquids into the distillation system. In an embodiment, the controller 151 is also coupled to the alignment system 501 shown in FIG. 5 as well as the rotational locking mechanism for the distillation tube 131 such as a solenoid 509 actuated to retract the rod during the vertical alignment process, as shown in FIG. 6. The controller 151 is also coupled to the thermoelectric mechanism 113 as well as the pump 119, a thermoelectric radiator 117 and a temperature transducer 905 that monitors the internal temperature of the fermentation tank. The temperature transducer and heater system 531 used to protect the membrane from thermal damage can also be coupled to the controller 151 so that the system can prevent thermal damage to the membrane as described above. An ultrasonic sensor 535 coupled to the ethanol tank is also in communication with the controller 151. The controller 151 is also coupled to the pumps 149 that control the flow of liquids from the ethanol and gas tanks.

Since the inventive micro refinery is intended to be a consumer product, at least a portion of the device may be collapsible. The ethanol fermentation tank is the largest component of the system and an empty volume before use. Thus, in an embodiment the fermentation tank is collapsible so that the system can be shipped in a reduced volume box or container. By collapsing the fermentation tank during transportation, storage space is minimized and shipping costs for delivery to customers and distributors is reduced. The collapsible fermentation tank can be placed on a rigid planar surface supported by the frame so that the weight of the fluids in the fermentation tank is supported while the tank provides structural support for the walls. There are various ways to make a collapsible fermentation tank.

In an embodiment, the tank is inflatable and can include one or more separate air chambers which each include an air or nitrogen gas valve that allows the user to inflate the chambers to a desired internal pressure. The air pressure and the tension of the walls create a fermentation tank structure that can securely contain the weight of the liquids placed in the tank. In this embodiment, the tank walls are preferably made of a flexible material that does not stretch in tension so that when inflated, thus the internal pressure causes the chamber walls to become fairly rigid. The walls have sufficient tensile strength to allow for the thermal expansion of the contained gas so the walls will remain inflated throughout all ambient temperature variations. When the air valves are opened and the chambers are deflated, the tank can be compressed into a much smaller volume.

In another embodiment, the tank can be made from one or more plastic panels that are more rigid than the inflatable embodiment. A plurality of wall panels that create the perimeter of the fermentation tank can be supported by posts that are mounted on the frame. In another embodiment, the fermentation tank may be made from a cylindrical piece of plastic material that is in tension when the interior is filled with the fermentation materials and water. In this embodiment, the cylindrical wall can be rolled for transportation. In other embodiment, the walls can have flexible joints or seams that allow the tank walls to be folded but still provide a liquid tight seal. In order to prevent leaks, a flexible membrane liner may be placed in the tank to form a water proof barrier over the base and around the side walls of the tank. The top of the fermentation tank is sealed and a lid allows access to the tank for cleaning and insertion of feedstock and sugar.

Since the tank will be used to ferment alcohol, the interior of the tank must be made from fuel compatible materials. In an embodiment, the interior of the fermentation tank is made of fuel compatible plastics and/or thin metal foil materials. In addition to being chemically compatible, the metal or metal alloy foil material may also allow the temperature of the internal liquid in the tank to be controlled by an outside temperature control system. Because foil is a good energy conductor, ambient heating or cooling of the internal volume of the tank from the outside can occur by conductive heat transfer rather than convection with the ambient air. Since the heating is done without exposure, the internal contents of the tank which are sensitive to biological virus cultures are not endangered by exposure to external biological contamination.

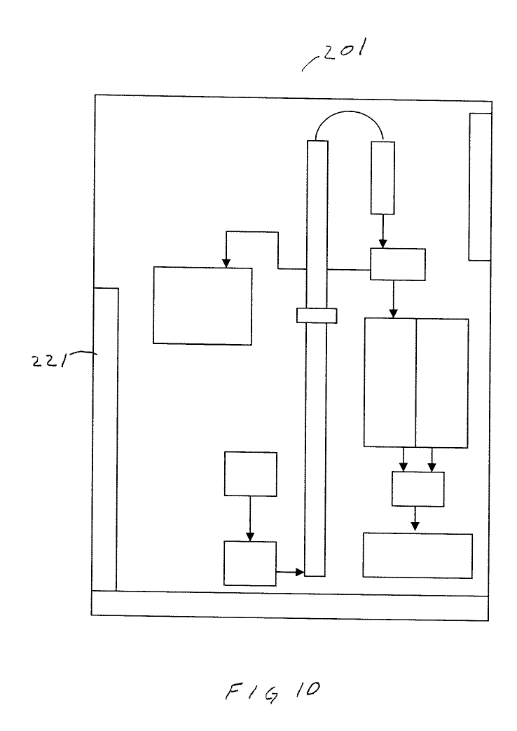

By providing a collapsible fermentation tank, the inventive micro refinery can be made smaller in volume for transportation. With reference to FIG. 10, in an embodiment the fermentation tank portion of the micro refinery system 201 is collapsible and the portion of the frame 221 normally under the fermentation tank can be folded against the housing of the other system components. This reduced size allows the system 201 to be more easily transported. The compressed fermentation tank can remain attached to the system 201 or alternatively, the tank can be separated and compressed in a separate protective container during transportation. During installation at the end user's site, the frame 221 can be folded down and the tank can be expanded, mounted on the frame 221 and connected to the rest of the system 201.

While the invention has been described herein with reference to certain preferred embodiments, these embodiments have been presented by way of example only, and not to limit the scope of the invention. Accordingly, the scope of the invention should be defined only in accordance with the claims that follow.

What is claimed is:

1. A micro refinery apparatus comprising:
a fermentation tank for processing a batch that includes water, sugar and yeast;
a thermoelectric mechanism coupled to the fermentation tank for heating and cooling the fermentation tank;
a temperature transducer coupled to the fermentation tank for detecting the temperature of the batch;
a CPU that receives temperature signals from the temperature transducer and transmits temperature control signals to the thermoelectric mechanism to maintain the temperature of the batch within a fermentation temperature range;
a distillation tube for coupled to the fermentation tank for distilling ethanol;
a gimbaled mechanism coupled to the distillation tube at a point above a center of gravity for allowing the distillation tube to rotate into vertical alignment; and
a porous membrane coupled to an outlet of the distillation tube for separating the water from ethanol.

2. The apparatus of claim 1, further comprising:
a storage tank for storing the ethanol;
a hose coupled to the storage tank;
a nozzle having a valve coupled to the hose;
an ethanol storage tank coupled to the porous membrane;
a first pump coupled between the ethanol storage tank and the hose;
a gasoline storage tank;
a second pump coupled between the ethanol storage tank and the hose;
wherein the CPU controls a flow rate of the ethanol through the first pump and a flow rate of gasoline through the second pump.

3. The apparatus of claim 1, further comprising:
an accelerometer for detecting gravitational information; and
a motorized alignment device for adjusting the rotation of the distillation tube;
wherein the CPU receives the gravitational information and determines a vertical direction and controls the alignment device to rotate the distillation tube into alignment with the vertical direction.

4. The apparatus of claim 1, further comprising:
an alignment device for adjusting the rotation of the distillation tube;
wherein the CPU receives the gravitational information and determines a vertical direction and controls the alignment device to rotate the distillation tube into alignment with the vertical direction.

5. The apparatus of claim 4, further comprising:
a locking device that prevents rotation of the distillation tube;
wherein the CPU actuates the locking device after the distillation tube has been aligned with the vertical direction.

6. The apparatus of claim 1, further comprising:
a load cell coupled to the fermentation tank for detecting a weight of the sugar in the fermentation tank;
a first valve coupled between a water source and the fermentation tank;
wherein the sugar is placed in the fermentation tank and the weight of the sugar is detected by the CPU before the water and yeast are added to the fermentation tank and the CPU controls the flow of the water through the first valve so that a volume of water corresponding to the weight of the sugar is added to the fermentation tank.

7. The apparatus of claim 6, further comprising:
a load cell coupled to the fermentation tank for detecting a weight of the sugar in the fermentation tank;
wherein the CPU monitors the weight of the batch over time and detects when the batch has been fully fermented.

8. The apparatus of claim 1,
wherein the fermentation tank is collapsible.

9. The apparatus of claim 1, further comprising:
a radiator pump coupled to the fermentation tank; and
a thermoelectric coupling radiator coupled to the radiator pump and the fermentation tank;
wherein the CPU actuates the radiator pump if the batch temperature is outside the fermentation temperature range and the CPU causes the thermoelectric coupling radiator to heat the batch if the batch temperature is below the fermentation temperature range or causes the thermoelectric coupling radiator to cool the batch if the batch temperature is above the fermentation temperature range.

10. A micro refinery apparatus comprising:
a fermentation tank for processing a batch that includes water, sugar and yeast;
a thermoelectric mechanism for heating and cooling the batch;
a temperature transducer coupled to the fermentation tank for detecting the temperature of the batch;
a distillation tube for coupled to the tank for distilling ethanol; and
a CPU coupled to the temperature transducer and the load cell for controlling the thermoelectric mechanism to maintain the temperature of the batch at within a fermentation temperature range.

11. The apparatus of claim 10, further comprising:
an accelerometer for detecting gravitational information; and
a motorized alignment device for adjusting the rotation of the distillation tube;
wherein the CPU receives the gravitational information and determines a vertical direction and controls the alignment device to rotate the distillation tube into alignment with the vertical direction.

12. The apparatus of claim 11, further comprising:
a locking device that prevents rotation of the distillation tube;
wherein the CPU actuates the locking device after the distillation tube has been aligned with the vertical direction.

13. The apparatus of claim 10, further comprising:
a radiator pump coupled between the fermentation tank and the thermoelectric mechanism; and
wherein the thermoelectric mechanism is a thermoelectric coupling radiator coupled to the radiator pump and the fermentation tank and the CPU actuates the radiator pump if the batch temperature is outside the fermentation temperature range and the CPU causes the thermoelectric coupling radiator to heat the batch if the batch temperature is below the fermentation temperature range or causes the thermoelectric coupling radiator to cool the batch if the batch temperature is above the fermentation temperature range.

14. The apparatus of claim 10, further comprising:
a load cell coupled to the fermentation tank for detecting a weight of the sugar in the fermentation tank;
a first valve coupled between a water source and the fermentation tank;
wherein the sugar is placed in the fermentation tank and the weight of the sugar is detected by the CPU before the water and yeast are added to the fermentation tank and the CPU controls the flow of the water through the first valve so that a volume of water corresponding to the weight of the sugar is added to the fermentation tank.

15. The apparatus of claim 14, further comprising:
a load cell coupled to the fermentation tank for detecting a weight of the sugar in the fermentation tank;
wherein the CPU monitors the weight of the batch over time and detects when the batch has been fully fermented.

16. The apparatus of claim 15, further comprising:
a second valve coupled to the fermentation tank; and
a heater coupled between the second valve and the distillation tube;
wherein the CPU controls the second valve so that liquids from the fermentation tank are vaporized by the heater before flowing through the distillation tube.

17. The apparatus of claim 10, wherein the fermentation tank is collapsible.

18. The apparatus of claim 10, further comprising:
an ethanol storage tank receiving the ethanol from the distillation tube;
a first pump coupled to the ethanol storage tank;
a gasoline storage tank;
a second pump coupled to the ethanol storage tank;
wherein the CPU controls a flow rate of ethanol through the first pump and a flow rate of gasoline through the second pump.

19. The apparatus of claim 17 further comprising:
a user interface coupled to the CPU for setting a ratio for the flow rate of the ethanol to the flow rate of the gasoline.

20. A micro refinery apparatus comprising:
a fermentation tank for processing a batch that includes water, sugar and yeast;
a thermoelectric mechanism for heating and cooling the fermentation tank;
a temperature transducer coupled to the fermentation tank for detecting the temperature of the batch;
a porous membrane receiving fluid from the fermentation tank and separating the water from ethanol; and
a CPU coupled to the temperature transducer and the load cell for controlling the thermoelectric mechanism to maintain the temperature of the batch at within a fermentation temperature range.

21. The apparatus of claim 20, further comprising:
a storage tank for storing the ethanol;
a hose coupled to the storage tank; and
a nozzle having a valve coupled to the hose.
an ethanol storage tank coupled to the porous membrane;
a first pump coupled between the ethanol storage tank and the hose;
a gasoline storage tank;
a second pump coupled between the ethanol storage tank and the hose;
a CPU for controlling a flow rate of ethanol through the first pump and a flow rate of gasoline through the second pump.

22. The apparatus of claim 20, further comprising:
a load cell coupled to the fermentation tank for detecting a weight of the sugar in the fermentation tank;
a first valve coupled between a water source and the fermentation tank;
wherein the sugar is placed in the fermentation tank and the weight of the sugar is detected by the CPU before the water and yeast are added to the fermentation tank and the CPU controls the flow of the water through the first valve so that a volume of water corresponding to the weight of the sugar is added to the fermentation tank.

23. The apparatus of claim 10, further comprising:
a load cell coupled to the fermentation tank for detecting a weight of the batch in the fermentation tank;
wherein the CPU monitors the weight of the batch over time and detects when the batch has been fully fermented.

24. The apparatus of claim 20, further comprising:
a membrane temperature transducer coupled to the CPU; and
a membrane heater;
wherein the CPU detects a temperature of the membrane and causes the membrane heater to heat the membrane if the temperature is below a thermal damage temperature.

25. The apparatus of claim 20, wherein the fermentation tank is collapsible.
